(19) United States
(12) Patent Application Publication
(10) Pub. No.: US 2004/0133141 A1
(43) Pub. Date: Jul. 8, 2004

(54) Wound dressing

(76) Inventors: Andrea Kiel, TM Utrecht (NL); Elke Leuchten, Krefeld (DE); Thomas Wolf, Hohr-Grenzhausen (DE); Thomas Pontzen, Koln (DE)

Correspondence Address: Friedrich Kueffner, Suite 910, 317 Madison Avenue, New York, NY, 10017, US

(21) Appl. No.: 10/468,305

(22) PCT Filed: Feb. 18, 2002
(86) PCT No.: PCT/EP02/01713

(51) Int. Cl. A61F 13/00; A61F 15/00

(52) U.S. Cl. 602/41

(57) ABSTRACT

The invention relates to a wound dressing, in particular a wound compress, comprising a layer containing active charcoal, at least one further layer, such as a sleeve for the active charcoal containing layer, an absorbent layer, a clothes protection layer, or similar and an agent with anti-microbial effect, characterised in that the agent with anti-microbial effect is contained in the at least one further layer.

The invention concerns a wound dressing, especially a wound compress, which comprises a layer that contains activated carbon, at least one other layer, such as a cover layer for the layer that contains the activated carbon, an absorbent layer, a layer for protecting the clothing, or the like, and an antimicrobial agent.

Wound dressings of this type, in the form of surgical dressings or tampons, are described, for example, in EP 0 053 936 B1. In the products described in the cited document, the layer that contains the activated carbon is used to adsorb malodorous substances and microorganisms and therefore contributes to a reduction of the risk of infection in the treatment of wounds. In this regard, the activated carbon layer of the previously known wound dressing may be present in the form of an activated carbon substance and be arranged between two cover layers. To achieve further reduction of the risk of infection, in the product described in the cited document, an antimicrobial agent is adsorbed on the layer that contains the activated carbon and is present in an amount that saturates no more than 20% of the adsorbent sites that are capable of adsorbing the antimicrobial agent. According to the cited document, antimicrobial agents that may be used are iodine, chlorinated phenols, antibiotics, and the like. The disclosed content of EP 0 053 936 B1 with respect to the antimicrobial agents and activated carbon layer described therein is herewith incorporated in the present specification by explicit reference.

A problem that has been encountered with the product known from the cited document is that the layer containing the activated carbon is already covered to a large extent by the antimicrobial agent, so that it is no longer fully available for the adsorption of malodorous substances, bacteria, or the like.

To solve this problem, WO 86/05,970 proposes a wound dressing with a layer that contains activated carbon but no antimicrobial agent. In this wound dressing, the risk of infection is reduced by providing, in addition to the layer containing the activated carbon, another layer made of a permeable material and a layer made of a semipermeable material on the outside of the dressing. Together, these two layers have antibacterial properties, because the growth of the bacteria adsorbed on the layer containing the activated carbon is inhibited by restriction of the oxygen supply, and the additional layers provide a barrier to bacteria, thereby preventing contamination from the outside. The cited document also describes a method for producing a layer containing activated carbon, in which a flat textile material is produced by impregnating suitable fibers with a solution of inorganic halogen compounds and then heating them under controlled conditions to effect the activation. The disclosed content of WO 86/05,970 with respect to the layers containing activated carbon and their production, which are described therein, is herewith incorporated in the present specification by explicit reference.

Finally, still another wound dressing that has a layer containing activated carbon is described in EP 0 311 364, in which at least 10% of the pore volume of the activated carbon is present in the form of mesopores. The disclosed content of EP 0 311 364 is also herewith incorporated in the present specification by explicit reference.

Although the risk of infection in the treatment of wounds can be significantly reduced with the products described above, it has been found that, even with the use of layers containing activated carbon, including those impregnated with an antimicrobial agent, wound infections occur again and again.

In light of the above-described problems associated with the state of the art, the object of the invention is to make available a wound dressing, especially a wound compress, with which the risk of infection in the treatment of wounds can be further reduced.

In accordance with the invention, this object is achieved by a modification of a wound dressing of the type mentioned at the beginning, for example, the wound dressing known from EP 0 053 936 B1. This modification is essentially characterized by the fact that the antimicrobial agent is contained in the at least one additional layer.

This invention is based on the surprisingly simple recognition that the antimicrobial properties of an antimicrobial agent can still be utilized in a meaningful way, even when this agent is not applied directly to the layer containing the activated carbon, which adsorbs the bacteria, but rather is contained in another, separate layer of the wound compress, so as to make available the full adsorption capacity of the layer that contains the activated carbon for the adsorption of malodorous substances, bacteria, and the like.

In this regard, the additional layer that contains the antimicrobial agent may be present in the form of a flat textile material, especially a nonwoven fabric or a woven or knitted fabric. A flat textile material of this type can be provided with the antimicrobial agent by impregnation or by the application of a suitable finish, especially a secondary finish. Alternatively or additionally, however, it is possible to produce the flat textile material from filaments, fibers and/or yarns that already contain the antimicrobial agent. If these filaments, fibers and/or yarns are polymers, the antimicrobial agent can be introduced into them before and/or during the polymerization. In this way, textiles with antimicrobial properties are obtained, which are permanently fixed in the textile and cannot be washed out.

Additionally or alternatively to a flat textile material that contains the antimicrobial agent, the wound dressing of the invention may also contain another layer in the form of a foil that contains the antimicrobial agent. This foil and/or the flat textile material containing the antimicrobial agent can be joined, especially with adhesives and/or by needling, with the layer containing the activated carbon or with another layer of the wound dressing, such as an absorbent layer, a cover layer or the like, so as to ensure a permanent positional relationship between the layer containing the activated carbon and the layer containing the antimicrobial agent and to achieve an especially reliable reduction of the risk of infection.

In this regard, the antimicrobial agent may be released in doses from the at least one additional layer and/or it may act by surface contact. In the case of dosed release, it is especially preferred for the antimicrobial agent to be released in low, but still sufficient, concentration over a period of several days. In a preferred embodiment of the invention, metals or metal ions are used as antimicrobial agents, and the use of silver or silver ions is especially preferred. In this regard, the antimicrobial agent may contain a silver zeolite, a crystalline material, such as silver zirconium phosphate, or even an amorphous, glassy compound that releases silver ions.

If the layer containing the activated carbon is covered on at least one side by a cover layer, such as a cover layer consisting of a nonwoven material, the antimicrobial agent may be contained in the cover layer and/or a layer which is arranged between the cover layer and the layer containing the activated carbon and which is preferably joined with the layer containing the activated carbon. If large amounts of wound secretions are to be absorbed by the wound dressing of the invention, as in the case of wound dressings intended for the covering of surface wounds, it has been found to be especially advantageous to provide an absorbent layer on the side of the layer containing the activated carbon which faces away from the layer containing the antimicrobial agent. In this regard, contamination of the area around the wound, especially the clothing in the vicinity of the wound, can be prevented by additionally providing a layer for protecting the clothing on the side of the absorbent layer which faces away from the layer containing the activated carbon.

The anitmicrobial agent of the wound dressing of the invention may contain chitin, chitosan, trichlosan and/or chlorhexidine.

The invention is explained below with reference to the drawings, which reveal all essential details of the invention not described above.

FIG. 1 shows a schematic representation of a first embodiment of the invention.

FIG. 2 shows a schematic representation of a second embodiment of the invention.

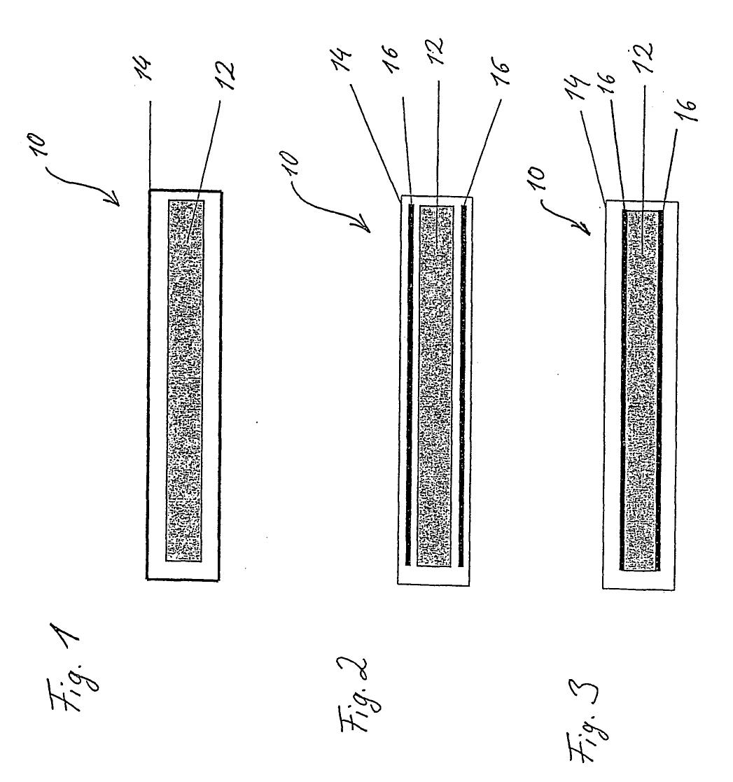

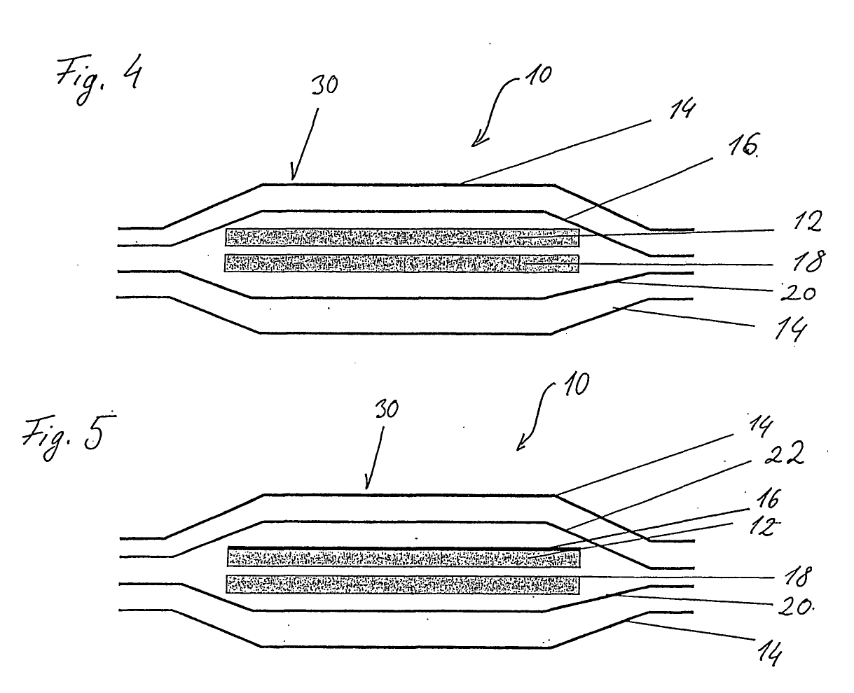

FIG. 3 shows a schematic representation of a third embodiment of the invention.

FIG. 4 shows a schematic representation of a fourth embodiment of the invention.

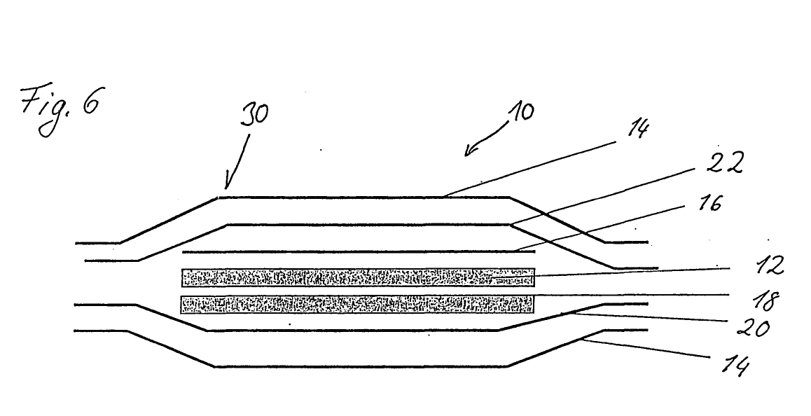

FIG. 5 shows a schematic representation of a fifth embodiment of the invention.

FIG. 5 shows a schematic representation of a sixth embodiment of the invention.

In the following explanation of preferred embodiments of the invention, the same reference numbers are used to label corresponding elements of the wound dressing of the invention in different drawings.

The wound compress 10 shown in FIG. 1 consists essentially of a flat structure 12 that contains activated carbon and a cover layer 14 that completely surrounds this flat structure 12 and may be in the form of a covering material made of nonwoven fabric. In the wound compress in accordance with FIG. 1, the antimicrobial agent is contained in the cover layer 14.

The embodiment of the invention shown in FIG. 2 also comprises a flat structure 12 that contains activated carbon and a cover layer 14. In this embodiment, the antimicrobial agent is contained in two additional layers 16 arranged on opposite sides of the flat structure 12 that contains the activated carbon between the flat structure 12 that contains the activated carbon and the cover layer 14. The embodiment of the invention shown in FIG. 3 differs from the embodiment illustrated in FIG. 2 essentially only in that the layers 16 that contain the antimicrobial agent are joined with the flat structure that contains the activated carbon, especially with adhesives or by needling.

All of the embodiments of the invention illustrated in FIGS. 1 to 3 can be used on both sides. The wound compress 10 shown in FIG. 4 comprises a flat structure 12 that contains the activated carbon and a cover layer 14 that covers this flat structure on both sides. On one side of the flat structure 12 that contains the activated carbon, a flat structure 16 that contains an antimicrobial agent is arranged between the flat structure 12 that contains the activated carbon and the cover layer 14. On the opposite side of the flat structure 12 that contains the activated carbon from the side covered by the layer 16 that contains the antimicrobial agent, an absorbent layer 18 and a clothing protection layer 20 are arranged between the flat layer 12 that contains the activated carbon and the cover layer 14.

The embodiment of the invention shown in FIG. 5 differs from the embodiment shown in FIG. 4 essentially only in that the flat structure that contains the antimicrobial agent is joined with the flat structure that contains the activated carbon, especially with adhesives or by needling. Furthermore, in the embodiment of the invention shown in FIG. 5, an anti-trickle layer 22 is provided between the flat structure that contains the antimicrobial agent and the cover layer 14, which prevents components that contain activated carbon from the flat structure that contains the activated carbon from permeating into the wound.

The embodiment of the invention shown in FIG. 6 represents a combination of the embodiments illustrated in FIGS. 4 and 5, in which the flat structure that contains the antimicrobial agent is not joined with the flat structure that contains the activated carbon, and an anti-trickle layer 22 is provided between the cover layer 14 and the flat structure that contains the antimicrobial agent.

The embodiments of the invention illustrated in FIGS. 4 to 6 can be used on only one side. The wound side is labeled with reference number 30.

The invention is not limited to the embodiments illustrated in the drawings, but rather the use of bandage-like wound dressings or wound dressings suitable for the tamponade of deep wounds is also possible. Furthermore, the wound dressing of the invention may also be provided with additional functional layers.

1. Wound dressing, especially a wound compress, which comprises a layer (12) that contains activated carbon, at least one other layer, such as a cover layer (14) for the layer (12) that contains the activated carbon, an absorbent layer (18), a layer (20) for protecting the clothing, or the like, and an antimicrobial agent, characterized by the fact that the antimicrobial agent is contained in the at least one additional layer (16).

2. Wound dressing in accordance with claim 1, characterized by the fact that the at least one additional layer (16) is present in the form of a flat textile material, especially a nonwoven fabric or a woven or knitted fabric.

3. Wound dressing in accordance with claim 2, characterized by the fact that the flat textile material (16) is impregnated with the antimicrobial agent.

4. Wound dressing in accordance with claim 2 or claim 3, characterized by the fact that the antimicrobial agent is applied as a finish to the flat textile material.

5. Wound dressing in accordance with any of claims 2 to 4, characterized by the fact that the flat textile material has filaments, fibers and/or yarns that contain the antimicrobial agent.

6. Wound dressing in accordance with claim 5, characterized by the fact that the filaments, fibers and/or yarns consist of polymers, and the antimicrobial agent is introduced into the filaments, fibers and/or yarns before the polymerization.

7. Wound dressing in accordance with any of the preceding claims, characterized by the fact that the at least one additional layer has a foil that contains the antimicrobial agent.

8. Wound dressing in accordance with any of claims 2 to 7, characterized by the fact that the foil and/or the flat textile material is joined, especially with adhesives and/or by needling, with the layer that contains the activated carbon and/or with another layer of the wound dressing, such as an absorbent layer (18).

9. Wound dressing in accordance with any of the preceding claims, characterized by the fact that the antimicrobial agent is released in doses from the at least one additional layer.

10. Wound dressing in accordance with any of the preceding claims, characterized by the fact that the antimicrobial agent is effective by contact with the surface.

11. Wound dressing in accordance with any of the preceding claims, characterized by the fact that that the antimicrobial agent contains silver and/or silver ions.

12. Wound dressing in accordance with any of the preceding claims, characterized by the fact that the antimicrobial agent contains silver zeolite, a crystalline material, such as silver zirconium phosphate, and/or a glassy compound.

13. Wound dressing in accordance with any of the preceding claims, characterized by the fact that the layer (12) containing the activated carbon is covered on at least one side by a cover layer (14), such as a cover layer consisting of a nonwoven material.

14. Wound dressing in accordance with any of the preceding claims, characterized by the fact that the antimicrobial agent is contained in the cover layer (14).

15. Wound dressing in accordance with claim 13 or claim 14, characterized by the fact that the antimicrobial agent is contained in a layer (16) which is arranged between the cover layer (14) and the layer (12) containing the activated carbon and which is preferably joined with the layer containing the activated carbon.

16. Wound dressing in accordance with claim 14 or claim 15, characterized by an absorbent layer (18) and/or a clothing protection layer (20) arranged on the opposite side of the layer (12) containing the activated carbon from the side covered by the layer (16) containing the antimicrobial agent.

17. Wound dressing in accordance with any of the preceding claims, characterized by the fact that the antimicrobial agent contains chitin, chitosan, trichlosan, and/or chlorhexidine.
